(19) United States
(12) Patent Application Publication
(10) Pub. No.: US 2010/0057022 A1
(43) Pub. Date: Mar. 4, 2010

(54) Method and Device for the Cleaning and Medical Treatment of Extremities

(76) Inventor: David John Horrigan, Encinitas, CA (US)

Correspondence Address: HORRIGAN CORPORATION, 2300 EIGHTH ST., OLIVENHAIN, CA, 92024, US

(21) Appl. No.: 12/038,727

(22) Filed: Feb. 27, 2008

(51) Int. Cl. A61M 35/00

(52) U.S. Cl. 604/289

(57) ABSTRACT

The use of elastomeric gloves, slippers, finger and toe covers for the cleaning, debriding and application of medicines by utilizing the repeated self-contained flushing and pumping caused by normal appendage motion and elastomeric distortion and equalization either alone or in tandem with the auxiliary agitation from an external ultrasonic source if necessary.

### CROSS-REFERENCE TO RELATED APPLICATIONS

Ser. No. 11/747,997

### SEQUENCE LISTING OF PROGRAM

Not Applicable

### FIELD OF THE INVENTION

This invention relates to the cleaning of and/or medical treatment of nails and skin located on the extremities, specifically for the cleaning and treatment of disorders that are difficult to treat due to an inability to keep remedying fluids on the infected, contaminated or damaged site.

### PRIOR ART

Pat. No. 7,137,148 PVC Based Medical Gloves and Formulations Therefor Pat. No. 7,056,309 Hygienic Cleansing Aid Pat. No. 6,931,767 Foot Cover Pat. No. 6,905,487 Dermal Treatment Applicator and Method Relating thereto Pat. No. 6,953,582 Skin-enhancing Glove and Method of Manufacture Pat. No. 6,913,758 Antimicrobial Glove and Method of Making Same Pat. No. 6,673,054 Body Protection Article Having a Gelatinous Material with a Therapeutic Additive Pat. No. 6,117,119 Gelatinous Body Protection Article Having a Therapeutic Additive Pat. No. 5,891,116 Hot Wax Skin Treatment Pat. No. 5,614,202 Moisturizing Glove Pat. No. 4,908,881 Finger Guard Pat. No. 4,087,675 Heated Mitten

### BACKGROUND OF THE INVENTION

Presently the application of medicines to the extremities is restricted to thick ointments supported by a bandage covering the afflicted area. The bandages wick the medicines away from the wound in short order making the medicines ineffective. Cleaning these extremities can be an equally complex task as areas such as nail undersurfaces are difficult to reach. The best method discovered thus far is scrubbing with a solvent and stiff brush. This cleaning method is time-consuming and has limited reach while the medicine application methods provide limited medicine contact with the wound.

Different inventors over the years have utilized complex elastomeric gloves or slippers for the application of impregnated moisturizer or wax but these require a dedicated and expensive device for application of their technology.

The inventor noticed that nails, especially diseased nails, offered some very difficult cleaning and treatment challenges as the debris and keratin buildup under the nail was tough and unreachable by existing cleaning techniques. Additionally, treating infections under the nail required both the removal of this debris and the recirculation of fresh medicines into the cavity under the nail.

### SUMMARY OF THE INVENTION

Accordingly it is a principal object of the present invention to overcome the disadvantages associated with prior art methods and systems and provide a simple yet effective way to thoroughly clean and/or apply medicine to nails, fingers, toes, feet and hands by use of an elastomeric sheath partially filled with cleaning solvent or medicine and fitted over the extremity being treated. Normal motion of the extremity will cause the fluid to slosh, shift and squirt within the sheath forcing the fluids into any cavities within the sheath and around any debris and infectious agents, flushing them out or continuously redepositing fresh solvent on the hidden surfaces within the site much like surf washes the shoreline.

From a medical point of view, it is another object of the invention to facilitate the continuous reapplying of a single dose of medicine via this pumping action which serves to make the single dose more effective than if the dose was statically applied as on a bandage or by way of a cream as in those instances only a small percentage of the medicine actually contacts the wound or contaminated site. If the fluids are more liquid to overcome this problem then they simply run off the wound and are absorbed into the bandage or local clothing. The sloshing motion of the medicine within the sheath of the invention continuously exposes the treatment site with new medicine surface that hasn't been depleted or contaminated.

From a cleaning point of view, it is yet another object of the present invention to provide exceptional cleaning of the extremities and the complex surfaces thereon by having the solvents continuously forced into and around areas that cannot otherwise be accessed easily and allows time for debris buildup to soften and be flushed away without the extensive labour costs associated with having a technician accomplish the same task with metal probes and scrapers.

It is a further object of the present invention to provide a vehicle that also allows solvent or medicine access to areas that may not be comfortably accessible by other methods of cleaning, debriding or treatment.

### BRIEF DESCRIPTION OF THE DRAWING-FIGURES

For a better understanding of the cleaning and medicinal applications of this invention with regard to the embodiments thereof, reference is made to the accompanying drawings, in which like numerals designate corresponding elements or sections throughout, and in which:

FIG. 1. illustrates a section of a toe or finger end with nail having a disease cavity and a buildup of keratin under the distal end of the nail plate.

FIG. 2. is a section of a toe or finger end with nail having a disease cavity and a buildup of keratin under the distal end of the nail plate. The toe or finger is covered with a elastomeric covering and that covering contains a quantity of cleaning fluid or medicine.

FIG. 3. is a male form for forming the sheaths.

### DRAWINGS-REFERENCE NUMBERS

Reference number 4, illustrates a keratin dam buildup caused by fungal waste and spores.

Reference number 5, shows a cavity created by the separating of the nail plate from the nail bed by the growing dam.

Reference number 6, shows the flushing fluid contained in proximity to the build-up and fungal infection

Reference number 7, shows the elastomeric covering of the finger or toe.

Reference number 8, shows the rolled collar that secures the integrity of the fluid container.

Reference number 9, shows the solvent or medicine within the cavity being cleansed or healed.

Reference number 10, shows the nail plate.

Reference number 11, shows groves cut in the form to add fluid-tightness.

Reference number 12, shows the shape of the form that gives the sheath its fluid directing flow.

Reference number 13, Shows the lofted sections that give the sheath its unique pumping shape.

### DETAILED DESCRIPTION OF THE PREFERRED EMBODIMENT

As required, detailed embodiments of the present invention are disclosed herein, however, it is to be understood that the disclosed embodiments are merely exemplary of medical or cosmetic applications of the invention which may be additionally embodied in various forms encompassing an expert diagnosis and supervision in different treatment requirements. Therefore, specific structural and functional details disclosed herein are not to be interpreted as limiting, but merely as a basis for the claims and as a representative basis for teaching one skilled in the art to variously employ the present invention in virtually any appropriately detailed situation.

Reference is now made to the drawings, wherein like characteristics and features of the present invention shown in the various FIGURES are designated by the same reference numerals.

Referring to FIGS. (1), (2), and (3) illustrating the preferred embodiment for the invention:

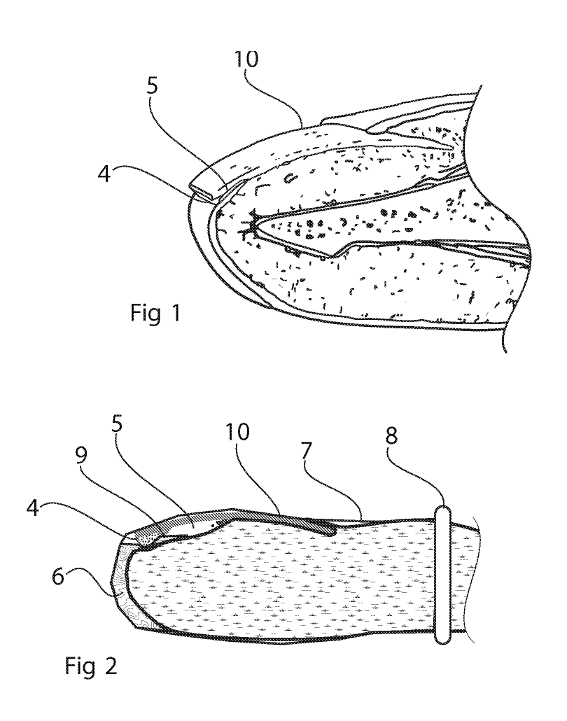

FIG. 1 is a section of a toe or finger end with nail having a disease cavity and a buildup of keratin under the distal end of the nail plate.

FIG. 2. is a section of a toe or finger end with nail having a disease cavity and a buildup of keratin under the distal end of the nail plate. The toe or finger is covered with a elastomeric covering and that covering contains a quantity of cleaning fluid or medicine.

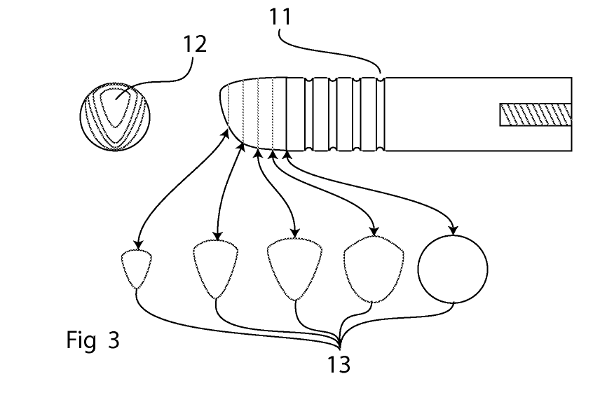

FIG. 3. shows a machined form for shaping the elastomeric sheath.

Reference number 4, illustrates a keratin dam buildup caused by fungal waste and spores.

Reference number 5, shows a cavity created by the separating of the nail plate from the nail bed by the growing dam.

Reference number 6, shows the flushing fluid contained in proximity to the build-up and fungal infection.

Reference number 7, shows the elastomeric covering of the finger or toe.

Reference number 8, shows the rolled collar that secures the integrity of the fluid container.

Reference number 9, shows the solvent or medicine within the cavity being cleansed or healed.

Reference number 10, shows the nail plate.

Reference number 11, shows groves cut in the form to add fluid-tightness.

Reference number 12, shows the shape of the form that gives the sheath its fluid directing flow.

Reference number 13, shows the lofted sections of the form that give the sheath its unique shape.

### FACTORS FOR EFFECTIVENESS

In order to make the invention as effective as possible there are three important factors that need to be taken into account. Firstly, the skins or sheaths must be impervious to the solvents and medicines it is expected to distribute.

Secondly, the skins must fit snugly at the collar to prevent fluid loss, while being loose about the area surrounding the treatment location leaving room for sloshing, flushing and recirculating of the fluids. A ribbed area can also accomplish this sealing task with out using as much pressure on the appendage.

Thirdly, the time duration the sheath is worn should be appropriately long to accomplish complete remediation of the situation but not so long as to cause a sensitivity to the agents cleaning or medicating the site.

### ULTRASONIC ASSISTANCE

As effective as this elastomeric pumping delivery/cleaning system is, occasionally the person using the device may not be ambulatory or not be able to walk about and provide the pressures to make the device as effective as they need it to be. In this case the addition of an external ultrasonic tank would be used to provide the stimulation necessary to agitate the medicines or solvents within the cavities being treated. This would include individual finger/toe sized ultrasonic chambers as well as large tanks in which the patient could submerge the entire hand or foot. If the medicines were expensive the patient would wear the sheath filled with the medicine while the tank would be filled with an acoustic energy transmitting medium such as water. When using the ultrasonic assistance the finger or toe should be pointed down so that contaminants could fall from the cavity utilizing gravity to help remove the debris.

### SCOPE OF THE INVENTION

Thus the reader will see that this method and device creates an efficient, effective tool for cleaning, debriding and applying medicines to nails, fingers, toes, feet and hands in a way that will make the cleaning and medicine agents more effective while reducing the time and skill required to accomplish the same result using traditional methods for these tasks. While the above medical description contains many specificities, these should not be construed as limitations on the scope of the device and technique, but rather as an exemplification of one preferred embodiment thereof. Many other variations are possible. For example, the method could be applied to moisturizing, bleaching, pretreating for anticipated hazards, softening, toughening, osmotic medicating, partial and increased pressure environmental treatments, the addition of heat or cold to this method, etc.

I claim:

1. A system for cleaning complex appendage surfaces comprising:
a. A formfitting elastomeric covering with an extended fluid-tight collar with
b. an appropriate dosage of cleaning solution or viscous solvent cream inserted therein; that is worn, and while worn, the normal motion of the appendage/elastomeric covering causes the flushing, cleaning action to the complex appendage surfaces.

2. A system for cleaning complex appendage surfaces as in claim 1 comprising:
a. A formfitting elastomeric covering with an extended fluid-tight collar with surrounded by an ultrasonic agitating source
b. an appropriate dosage of cleaning solution or viscous solvent cream inserted therein; that is worn, and while worn, the ultrasonic motion of the fluid within the elastomeric covering causes the flushing, cleaning action to the complex appendage surfaces.

3. A system for applying medicine to complex appendage surfaces comprising:
c. A formfitting elastomeric covering with an extended fluid-tight collar with
d. an appropriate dosage of fluid or semifluid medicine or antimicrobial inserted therein; that is worn, and while worn, the normal motion of the appendage/elastomeric covering causes the flushing, squirting, distribution action to reach the otherwise inaccessible complex appendage surfaces.

4. A system for for applying medicine to complex appendage surfaces as in claim 3 comprising:
a. A formfitting elastomeric covering with an extended fluid-tight collar immersed in an ultrasonic agitating chamber
b. an appropriate dosage of fluid or semifluid medicine or antimicrobial inserted therein; that is worn, and while worn, the ultrasonic motion of the fluid within the elastomeric covering causes the flushing, squirting, distribution action to reach the otherwise inaccessible complex appendage surfaces.
